(19) United States
(12) Patent Application Publication
(10) Pub. No.: US 2024/0245550 A1
(43) Pub. Date: Jul. 25, 2024

(54) STIMULATING DEVICE FOR HUMAN AND ELECTRIC THERMAL THERAPY DEVICE HAVING FUNCTION FOR THERMAL THERAPY TO TEMPOROMANDIBULAR JOINT

(71) Applicant: Young Woo CHUN, Seoul (KR)

(72) Inventor: Young Woo CHUN, Seoul (KR)

(21) Appl. No.: 18/018,273

(22) PCT Filed: Nov. 29, 2021
(86) PCT No.: PCT/KR2021/017750

§ 371 Date: Jan. 27, 2023

(30) Foreign Application Priority Data
Nov. 29, 2021 (KR) 10-2021-0166685

(51) Int. Cl. A61F 7/00

(52) U.S. Cl. CPC A61F 7/007; A61F 2007/0011; A61F 2007/0013; A61F 2007/0015

(57) ABSTRACT

Disclosed is a head-wearable stimulating device for human having a function for thermal therapy to temporomandibular joints, and an electric thermal therapy device. The stimulating device for human according to the present disclosure is a device having a function for thermal therapy to temporomandibular joints, and includes: a body configured to be worn on a head; and an electric heating main thermal therapy section disposed at the body to be able to perform thermal therapy on temporomandibular joints and generating heat using electricity. The stimulating device for human may further include a neck stimulation band having a neck thermal therapy section for performing thermal therapy on muscles at a neck, and disposed under the body to be able to be worn on a neck. Since the present disclosure performs thermal therapy on temporomandibular joints on a head, it is possible to stably apply stimulation for human including thermal therapy to target areas even if a user is moving. Further, since the present disclosure heats temporomandibular joint areas in an electric heating type, it is possible to remove a process of preheating a material such as silica gel in a thermal therapy pack using water or a microwave over a predetermined time before performing thermal therapy on temporomandibular joints. Further, it is possible to remove various inconveniences such as the difficulty in keeping/maintaining temperature accompanying use of existing thermal therapy packs or the problem of a skin burn.

### TECHNICAL FIELD

The present disclosure relates to a stimulating device for human and, in more detail, to a stimulating device for human having a function for thermal therapy to a temporomandibular joint, and an electric thermal therapy device applying thermal therapy to a temporomandibular joint area using electrically generated heat, that is, electrical heat.

### BACKGROUND ART

Temporomandibular joints are joints connecting a mandible (lower jawbone) and a temporal bone (upper jawbone) and are positioned right in front of both ears. The temporomandibular joints act as a central axis for all jaw motions and are supported by jaw muscles and ligaments, and discs between the temporomandibular joints function as cushions between bones. Further, when a problem is generated with such temporomandibular joints, it is called a temporomandibular disorder or a temporomandibular joint disorder.

A pain is a representative symptom of temporomandibular disorders and patients feel a pain in the temporomandibular joints and masticatory muscles right in front of both ears when masticating food or yawning. Furthermore, a click (joint noise) that is generated from temporomandibular joints while opening one's mouth may be another symptom of temporomandibular disorders. Also, when there is a temporomandibular disorder, movements of the mouth and jaw are limited (mandibular motion is limited).

Temporomandibular disorders described above can be classified into disc derangement disorder, disorders of jaw muscles, arthritis, luxation, ankylosis, etc. Disc derangement disorder means a state in which disc in a temporomandibular joint comes out of the original position, and in this case, there is no specific symptom except for a click at temporomandibular joints when a mouth is opened or closed in the early stage, but sometimes there may be a feeling of locking of the jaw as the disorder proceeds.

When a temporomandibular disorder further proceeds, the mouth is suddenly not opened and the patient feels a severe pain in the temporomandibular joints. When there is arthritis, which accompanies the pain in the joint area, it is difficult to masticate food or move the jaw, and sometimes a crepitus, a sound like grinding sand is generated. When arthritis gets worse, the temporomandibular joints stiffen, so the mouth cannot be fully opened and it is difficult to take food. Since temporomandibular joints and jaw muscles are closely connected with each other in terms of function, there is a high possibility that the joint disease and the jaw muscle disease come together, which are musclesplinting, local myalgia, myospasm, myofascial pain, myositis, muscle contracture, etc. Muscle splinting, local myalgia, and myofascial pain are easily generated when fatigue of muscles is accumulated, myositis is easily generated by an injury or infection, and myospasm is easily generated with centrally mediated pathways or if there is a problem with electrolyte metabolism, which can be greatly influenced by various endocrine functions or psychologic factors.

Further, when temporomandibular disorders accompany a muscular problem, it is difficult to open the mouth or masticate food due to a pain, and this pain spreads to the head, the neck, the shoulders, etc. in some cases.

There are various reasons that cause temporomandibular disorders, including bad habits such as bruxism, clenching, a bad sleep posture, chin buttressing, an injury, anxiety, tension, and depression due to stress, etc.

Since tissues constituting the temporomandibular joints are difficult to recover once injured, prevention and early treatment are the most important. It is the fundamental aim of treatment to protect temporomandibular joints from severe trauma in order to prevent the temporomandibular joints from being further damaged, and to stabilize the function, and thereafter, appropriate occlusal therapy, medication, physical therapy, exercise therapy, behavioral therapy, etc. are performed, depending on symptoms.

The occlusal therapy is also called splint therapy, and bruxism and clenching are important factors in temporomandibular joint diseases, so the occlusal therapy is a method of treatment for relaxing muscles in the head and neck area and by correcting positions of the structures by wearing a stabilizing device, that is, a splint over the teeth.

When a symptom of a temporomandibular disorder is severe, medication is applied, and nonsteroidal antiinflammatory drugs, a muscle relaxant, a tranquilizer, an antidepressant, etc. are prescribed. Further, it has been known that botulinum toxin injections that relax muscles are helpful when there is a severe muscle pain or an involuntary contracture.

Further, physical therapy and exercise therapy are required for muscle contracture, and pains are controlled through a moist hot or cold therapy, ultrasonic treatment, transcutaneous electrical nerve stimulation, and electroacupuncture stimulation therapy. The exercise therapy is a method to make temporomandibular joints comfortable and relax the muscles of head, neck, shoulders, and bio feedback therapy or surgical treatment can be applied to treatment of temporomandibular disorders besides the various temporomandibular treatment methods described above.

In particular, thermal therapy, which is widely used in physical therapy, is performed with a heated materials placed on the temporomandibular joint, so there are various inconveniences such as limiting the patient's activity, the material must be heated in advance, and if the heated material cools down over time during treatment, it must be reheated before use, it is difficult to manipulate the temperature of the heated material, so it is necessary to use it with a towel attached to prevent burns, and it is difficult to store after use due to its bulkiness. It is inconvenient and cumbersome to use because it has poor fit to the treating area and is heavy.

Accordingly, the inventor(s) has developed a stimulating device for human having a function for thermal therapy to temporomandibular joints, the stimulating device for human having a function for thermal therapy to temporomandibular joints, particularly, a function for thermal therapy, being able to perform thermal therapy on temporomandibular joints in the state in which it is worn on the head, and being able to remove inconvenience that it is required to heat a heat pack in advance using boiled water or a microwave, and in detail, the inventor(s) has developed an electric thermal therapy device that can be worn on the head.

### DISCLOSURE

### Technical Problem

An objective of the present disclosure is to provide a head-wearable stimulating device for human, for example, a headgear-type electric thermal therapy device that has a function for thermal therapy, particularly, a function for thermal therapy to temporomandibular joints, can be worn on the head, and can perform thermal therapy on temporomandibular joints using electrical heat.

Another objective of the present disclosure is to provide a stimulating device for human, for example, an electric thermal therapy device that can perform thermal therapy on head muscles such as the masseter muscles and/or temporalis muscles together with temporomandibular joints.

Another objective of the present disclosure is to provide a stimulating device for human, for example, an electric thermal therapy device that has a function for thermal therapy to neck muscles together with a function for thermal therapy to temporomandibular joints.

### Technical Solution

An aspect of the present disclosure provides a stimulating device for human having a function for thermal therapy to temporomandibular joints, which includes: a body configured to be worn on a head; and an electric heating main thermal therapy section disposed at the body to be able to perform thermal therapy on temporomandibular joints and generating heat using electricity.

It is preferable that the stimulating device for human according to an aspect of the present disclosure further includes at least one head muscle thermal therapy section of a masseter muscle thermal therapy section and a temporalis muscle thermal therapy section disposed at the body together with the main thermal therapy section.

The head muscle thermal therapy section may generate heat using electricity, for example, may be an electric heater. The main thermal therapy section and the head muscle thermal therapy section may be simultaneously operated or may be individually controlled to be able to independently operate.

The head muscle thermal therapy section includes a masseter muscle thermal therapy section and a temporalis muscle thermal therapy section, and the masseter muscle thermal therapy section and the temporalis muscle thermal therapy section may be individually controlled.

The body may include: a first body configured to be worn on a left side of a head from a center line; and a second body configured to be worn on a right side of a head from the center line.

The masseter muscle thermal therapy section may include: a left masseter muscle thermal therapy section disposed at the first body to perform thermal therapy on a left masseter muscle; and a right masseter muscle thermal therapy section disposed at the second body to perform thermal therapy on a right masseter muscle.

A lower limit of the left masseter muscle thermal therapy section may extend to a lower area of a left jaw line across a left lower edge of a lower jaw; and a lower limit of the right left masseter muscle thermal therapy section may extend to a lower area of a right jaw line across a right lower edge of a lower jaw. In more detail, the left masseter muscle thermal therapy section may be provided to an area covering a left lower edge of a lower jaw in a left cheek area in which a left masseter muscle is positioned. Further, the right masseter muscle thermal therapy section may be provided to an area covering a left lower edge of a lower jaw in a right cheek area in which a right masseter muscle is positioned.

The body may further include a connecting portion going through a pogonion while connecting the first body and the second body, and forming a non-heat generating region between the lower limit of the left masseter muscle thermal therapy section and the lower limit of the right masseter muscle thermal therapy section.

The temporalis muscle thermal therapy section may include: a left temporal thermal therapy section disposed at the first body to perform thermal therapy on a left temporalis muscle; and a right temporal thermal therapy section disposed at the second body to perform thermal therapy on a right temporalis muscle.

The main thermal therapy section may include: a first electric heating section disposed at the first body to perform thermal therapy on a left temporomandibular joint; and a second electric heating section disposed at the second body to perform thermal therapy on a right temporomandibular joint.

The first body may be worn on the left side of the head from the center line across any one area of the left temporalis muscle and the left masseter muscle, and a left temporomandibular joint; and the second body may be worn on the right side of the head from the center line across any one area of the right temporalis muscle and the right masseter muscle, and a right temporomandibular joint.

A first side of the first body and a first side of the second body may be detachably coupled so that the body is worn on a head. Further, the first body and the second body may be a structure having a flexible material.

The stimulating device for human may further include a neck stimulation band having a neck thermal therapy section for performing thermal therapy on muscles at a neck, and disposed under the body to be able to be worn on a neck. The neck stimulation band may be connected to the lower portion of the body.

The neck stimulation band may include: a left stimulation band being able to be worn on a left side of the neck; and a right stimulation band being able to be worn on a right side of the neck.

In order the put the neck stimulation band on a neck, a first side of the left stimulation band and a first side of the right stimulation band may be detachably connected, thereby forming a loop that is wound around a neck.

The neck thermal therapy section may generate heat to perform thermal therapy on at least one of a sternocleidomastoideole and a splenius capitis muscle of the neck. The neck thermal therapy section may include a first neck thermal therapy section for performing thermal therapy on the sternocleidomastoideole, and a second neck thermal therapy section for performing thermal therapy on the splenius capitis muscle. The first neck thermal therapy section and the second neck thermal therapy section may be individually or simultaneously controlled.

Another aspect of the present disclosure provides an electric thermal therapy device, that is, a head-wearable electric thermal therapy device having a function for thermal therapy to temporomandibular joints, which includes: a headgear configured to be worn on a head; a first electric heating section disposed at a first side of the headgear to be able to perform thermal therapy on a left temporomandibular joint at a left side of the head from a center line of the head, and generating heat using electricity; and a second electric heating section disposed at a second side of the headgear to be able to perform thermal therapy on a right temporomandibular joint at a right side of the head, and generating heat using electricity.

The electric thermal therapy device may further include: at least one electric heating left head muscle thermal therapy section of a left masseter muscle thermal therapy section and a left temporal thermal therapy section provided with the first electric heating section at the first side of the headgear; and at least one electric heating right head muscle thermal therapy section of a right masseter muscle thermal therapy section and a right temporal thermal therapy section provided with the second electric heating section at a second side of the headgear.

The electric thermal therapy device may further include a neck stimulation band having a neck thermal therapy section for performing thermal therapy on muscles at a neck, and disposed under the headgear to be able to be worn on the neck.

### Advantageous Effects

The head-wearable stimulating device for human having a function for thermal therapy to temporomandibular joints, and the electric thermal therapy device according to the present disclosure have the following advantages.

Since the present disclosure performs thermal therapy on temporomandibular joints on a head, it is possible to stably apply stimulation for human including thermal therapy to target areas even if a user is moving. Further, since the present disclosure heats temporomandibular joint areas in an electric heating type (electric heat generation type), it is possible to remove a process of preheating a material such as silica gel in a thermal therapy pack using water or a microwave over a predetermined time before performing thermal therapy on temporomandibular joints. Further, it is possible to remove various inconveniences such as the difficulty in keeping/maintaining temperature accompanying use of existing thermal therapy packs or the problem of a skin burn.

Second, according to the present disclosure, since it is possible to simultaneously perform thermal therapy on the temporomandibular joints, and the masseter muscles and the temporalis muscles, it is possible to more effectively alleviate temporomandibular disorders. Further, it is convenient to set and maintain temperature. Further, it is possible to implement a human stimulating device, in more detail, an electric thermal therapy device in various types such as simultaneously or individually controlling thermal therapy temperature and whether to perform thermal therapy on temporomandibular joint areas, masseter muscle areas, and temporalis muscle areas.

Third, according to the present disclosure, since it is possible to perform thermal therapy on neck muscles, in more detail, sternocleidomastoideoles and/or splenius capitis muscle together with temporomandibular joints, it is possible to attenuate a pain in masseter muscle or temporalis muscle areas, and temporomandibular joint areas.

Fourth, the stimulating device for human, in more detail, the electric thermal therapy device according to the present disclosure can be easily worn on a head using the detachable coupler that can be connected/disconnected, can stable transmit heat in close contact with thermal therapy object areas (target areas) such as and temporomandibular joint areas through a thin soft strap structure, and can be lightly and thinly manufactured, so it can be conveniently used.

### DESCRIPTION OF DRAWINGS

The features and advantages of the present disclosure will be more clearly understood from the following detailed description of embodiments of the present disclosure with the accompanying drawings to be described below, in which:

FIG. 1 is a planar view schematically showing an example of an electric thermal therapy device as an embodiment of a stimulating device for human according to the present disclosure;

FIG. 2 is a view showing a heat generation section of the stimulating device for human shown in FIG. 1;

FIG. 3 is a view showing an example of the structure of the heat generation section shown in FIG. 2;

FIG. 4 is a block diagram exemplifying a connection structure of a controller and the heat generation section shown in FIG. 3;

FIG. 5 is a side view showing a worn state of the stimulating device for human shown in FIG. 1;

FIG. 6 is a rear view showing the worn state of the stimulating device for human shown in FIG. 1;

FIG. 7 is a view showing the worn state of the stimulating device for human shown in FIG. 1 from under a jaw;

FIG. 8 is a view showing an example of a controller for an embodiment of the stimulating device for human according to the present disclosure;

FIG. 9 is a view exemplifying a temperature adjustment type and a time adjustment type by the controller shown in FIG. 8;

FIG. 10 is a view showing another example of the structure of the heat generation section shown in FIG. 2;

FIG. 11 is a view showing an example of a controller for controlling the operation of a stimulating device for human having the heat generation section of the structure shown in FIG. 10;

FIG. 12 is a planar view schematically showing another embodiment of a stimulating device for human according to the present disclosure;

FIG. 13 is a view showing a heat generation section of the stimulating device for human shown in FIG. 12;

FIG. 14 is a view showing an example of the structure of the heat generation section shown in FIG. 13;

FIG. 15 is a side view showing a worn state of the stimulating device for human shown in FIG. 12;

FIG. 16 is a rear view showing the worn state of the stimulating device for human shown in FIG. 12;

FIG. 17 is a view showing an example of a controller for controlling the operation of a stimulating device for human having the heat generation section of the structure shown in FIG. 13;

FIGS. 18 to 21 are planar views showing additional embodiments of the stimulating device for human according to the present disclosure;

FIG. 22 is a view exemplifying a worn state of another embodiment (mask type) of the stimulating device for human according to the present disclosure; and

FIG. 23 is a view exemplifying a worn state of another embodiment (hood type) of the stimulating device for human according to the present disclosure; and

### BEST MODE FOR INVENTION

Hereafter, an exemplary embodiment that can achieve the objectives of the present disclosure in detail is described with reference to the accompanying drawings. In the description of the embodiment, the same components are given the same names and reference numerals and are not repeatedly described.

The terminologies used herein are for the purpose of describing embodiments of the present disclosure only and are not intended to limit the present disclosure. For example, the terms including ordinal numbers such as “first” and “second” may be used to discriminate components having the same names, but are not intended to define or limit the numbers of the components.

It is to be understood that when one element is referred to as being “connected to” or “coupled to” another element, it may be connected directly to or coupled directly to another element or be connected to or coupled to another element with another element therebetween, including indirect connection.

It will be further understood that the terms “comprises” or “have” used in this specification, specify the presence of stated features, steps, operations, components, parts, or a combination thereof, but do not preclude the presence or addition of one or more other features, numerals, steps, operations, components, parts, or a combination thereof.

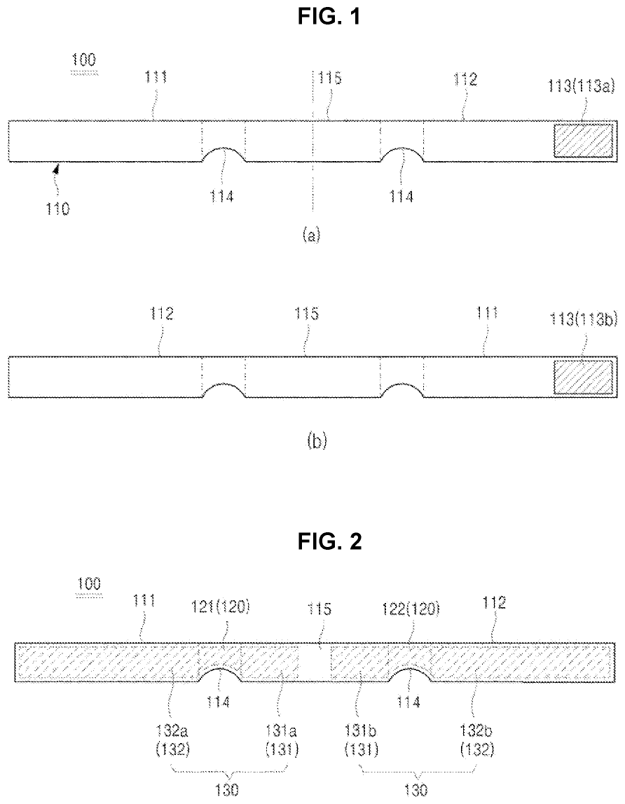

An embodiment (first embodiment) of a stimulating device for human according to the present disclosure is described first with reference to FIGS. 1 to 7, and the first embodiment of the stimulating device for human may be implemented as an electric thermal therapy device, in more detail, a head-wearable, in other words, headgear-type electric thermal therapy.

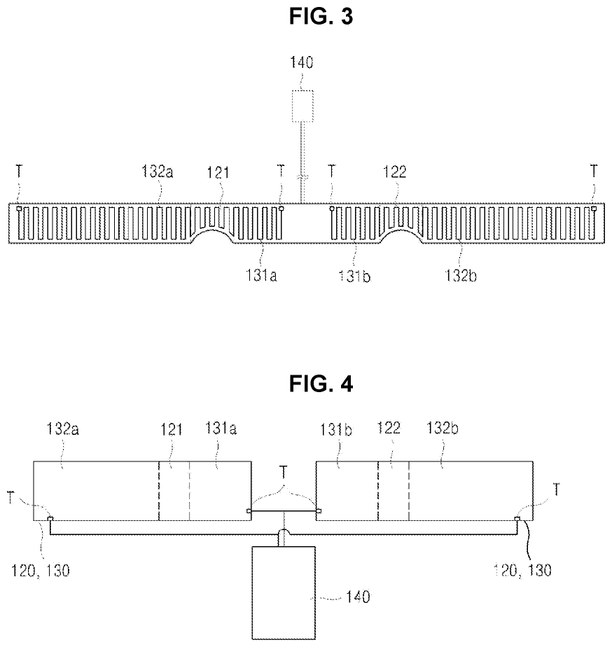

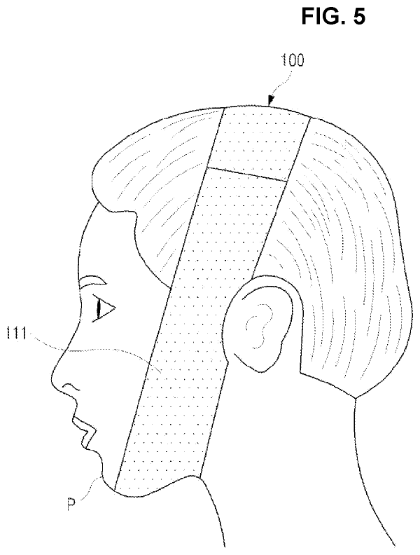

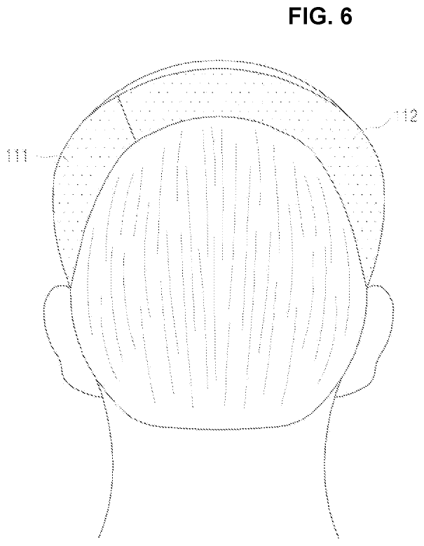

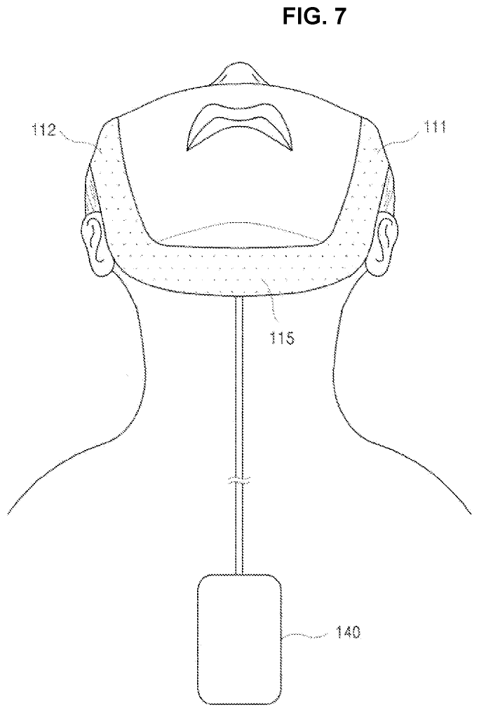

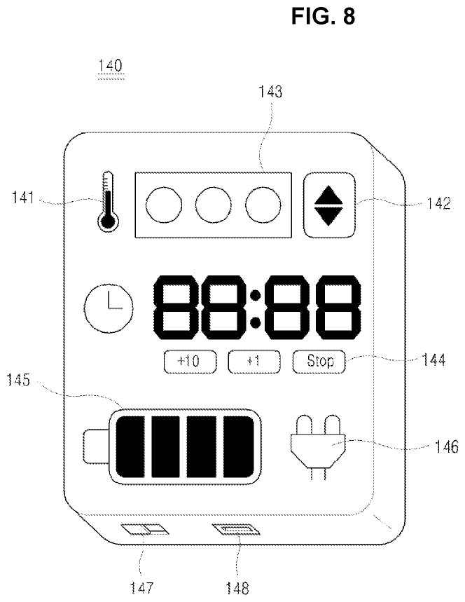

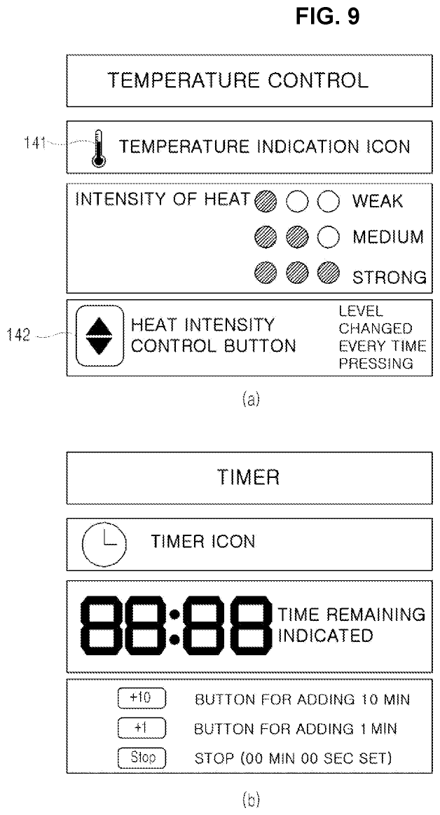

In figures for describing the first embodiment of the present disclosure, FIG. 1 is a planar view of an embodiment of a stimulating device for human, FIG. 2 is a view showing a heat generation section of the stimulating device for human shown in FIG. 1, FIG. 3 is a view showing an example of the structure of the heat generation section shown in FIG. 2, and FIG. 4 is a view showing an example of a controller for controlling the operation of the stimulating device for human shown in FIG. 1. Further, FIG. 5 is a side view (a left side view of a face) showing a worn state of the stimulating device for human shown in FIG. 1, FIG. 6 is a rear view (of the occiput) showing the worn state of the stimulating device for human shown in FIG. 1, and FIG. 7 is a view showing the worn state of the stimulating device for human shown in FIG. 1 from under a jaw.

Referring to FIGS. 1 to 7, a first embodiment of the stimulating device for human, which is a device supposed to be worn on a head and having a function for thermal therapy to temporomandibular joints, as will be described below, may be implemented as a headgear-type electric thermal therapy device.

A stimulating device for human 100 according to the first embodiment of the present disclosure, which is a device having a function for thermal therapy to temporomandibular joints, can alleviate temporomandibular disorders and attenuate a pain, and can alleviate facial muscle disorders or attenuate a pain of muscles related to temporomandibular joints.

The stimulating device for human 100 includes a body 110 for wearing on a head and an electric heating thermal therapy section 120 on the body. The electric heating thermal therapy section 120 is a component disposed on the body 100 to be able to perform thermal therapy on temporomandibular joints and generating heat using electricity, that is, an electric heating component (generating heat by converting electrical energy into heat energy). The electric heating thermal therapy section 120 having a thermal therapy section for temporomandibular joints is referred to as a main thermal therapy section hereafter to be discriminated from other thermal therapy sections to be described below.

A head muscle thermal therapy section 130 for performing thermal therapy on at least one kind of muscle of the masseter muscles and temporalis muscles may be additionally disposed on the body 110. In other words, the stimulating device for human 100 may further include at least a kind of head muscle thermal therapy section of a masseter muscle thermal therapy section 131 and a temporalis muscle thermal therapy section 132.

This is for more effectively alleviate temporomandibular disorders by performing thermal therapy on muscles that influence the function of the temporomandibular joints, particularly, the masseter muscles and/or the temporalis muscles. For reference, the masseter muscles and/or the temporalis muscles are muscles that are used to masticate food or close the mouth, the temporomandibular joints, which are areas connecting an upper jawbone and a lower jawbone and surrounded by fibrous capsules, are filled with a lubricant substance called synovia, and a disc is positioned between bones, and when inflammatory substances are accumulated at this sites, arthralgia and degenerative arthritis may be generated.

When an excessive force is applied to the temporomandibular joints and collagenous fibers of joint tissues are injured, the surfaces of the temporomandibular joints become rough, and when a mouth that has been closed for a while is opened (e.g., opened immediately after waking up in the morning), a click (joint noise) may be generated due to adherence or adhesion of the joint surfaces caused in this way. Further, when a ligament holding a disc of the temporomandibular joints is deformed and the disc comes out of the normal position, also, a click may be generated every time the jaw is moved, particularly, every time a mouth is opened or moved left and right.

Thermal therapy of temporomandibular joints can suppress joint noise that is generated in the jaw by suppressing damage to collagenous fibers of joint tissues and can suppress/attenuate the symptom of degenerative arthritis by removing inflammatory substances in the temporomandibular joints.

Thermal therapy of temporomandibular joints increases elasticity of the tissues of the temporomandibular joints, whereby stiffness of the temporomandibular joints decreases and movable range of the temporomandibular joints, that is, joint mobility can be increased. When the range of movement of temporomandibular joints increases, the mouth can be opened larger, and pumping that physically promotes metabolism at the joint is increased with movement of the jaw, which also contributes to recovery of temporomandibular disorders.

Next, the masseter muscles and the temporalis muscles are muscles positioned superficially, heat can be sufficiently transmitted to the masseter muscles and the temporalis muscles by thermal therapy and it is possible to obtain an effect of quickly attenuating a pain in the temporomandibular joints or muscles through thermal therapy. When there is a pain only in a joint due to inflammatory substances in the temporomandibular joints even if there is no problem with muscles such as the masseter muscles or the temporalis muscles, it is possible to reduce a pain in the temporomandibular joints by increasing blood circulation by performing thermal therapy on the masseter muscles and the temporalis muscles.

Thermal therapy of muscles such as the masseter muscles and/or the temporalis muscles increases blood circulation. It was reported by many studies that causes of muscle pain are associated with the state in which blood flow in tissues is reduced, and when heat is applied, the blood vessels of injured tissues dilate and blood circulation increases, so more oxygen and nutrients are supplied, whereby recovery can be promoted.

Further, thermal therapy of the masseter muscles and the temporalis muscles increases metabolism of the muscles, the fast metabolism promotes recovery, and heat increases elasticity of the muscles, whereby stiffness and soreness of the masseter muscles and the temporalis muscles are decreased, which positively influences reduction of a pain in the temporomandibular joint or alleviation of temporomandibular disorders. A pain may be attenuated even if blood circulation is not increased yet immediately after performing thermal therapy on the masseter muscles/the temporalis muscles, which can be explained by Gate control theory of a pain control mechanism.

The head muscle thermal therapy section 130 may be a thermal therapy pack that is heated by boiled water or a microwave unlike the main thermal therapy section 120, in more detail, may be a heating type thermal therapy pack that can be detachably attached to the body 110. In an embodiment of the present disclosure, the head muscle thermal therapy section 130 is a component that generates heat using electricity, for example, an electric heater such as a heating wire, and is an electric heating thermal therapy section like the main thermal therapy section 120.

Further, in the embodiment, the main thermal therapy section 120 and the head muscle thermal therapy section 130 are integrally operated. For example, the main thermal therapy section 120 and the head muscle thermal therapy section 130 may be formed in a heating wire structure connected in series or in parallel and integrally powered. Of course, as other examples to be described below, the main thermal therapy section 120 and the head muscle thermal therapy section 130 may be individually controlled to be able to independently operate.

In the embodiment, both the main thermal therapy section 120 and the temporalis muscle thermal therapy section 132 may be disposed at the body 110, and the masseter muscle thermal therapy section 131 and the temporalis muscle thermal therapy section 132 may also be integrally operated and may be individually controlled to be able to independently operate.

FIG. 3 shows a heating wire structure in which the main thermal therapy section 120, the masseter muscle thermal therapy section 131, and the temporalis muscle thermal therapy section 132 are connected in series, in which an integral control structure (integral operation structure) that forms an integrated electric heating thermal therapy section by the main thermal therapy section 120, the masseter muscle thermal therapy section 131, and the temporalis muscle thermal therapy section 132 is shown, and the names of the thermal therapy sections are given to separate regions for thermal therapy areas (target areas), respectively.

Referring to FIGS. 1 to 3, the body 110 includes a first body 111 supposed to be worn on the left side of a head from a center line and a second body 112 supposed to be worn on the right side of a head from the center line.

For reference, FIG. 1(a) is a planar view of the stimulating device for human seen from an inner side (a lining) of the body supposed to come in contact with a skin and FIG. 1(b) is a planar view showing the stimulating device of FIG. 1(a) turned over. The body 110 may be developed in a plane, but may have a curved shape, for example, a U-shape when bending is easy and an external force is not applied, or a shape of which the left-right distance increases toward the top, and the inner side of the body may be made of a spongy material so that the inner side of the body can be stably brought in close contact with a skin.

In more detail, the body 110, which is part supposed to be worn on a head, that is, a kind of headgear, may be formed in various types such as a soft band or strap type loop that is wound in the up-down direction around the left and right sides of a head, for example, diagonally wound around a head to cover a thermal therapy area when seen from a side of a face, or a mask type or a hood type covering a thermal therapy area.

The first body 111 may be worn on the left side of the head from the center line across any one area of the left temporalis muscle and the left masseter muscle, and the left temporomandibular joint area.

The second body 112 may be worn on the right side of the head from the center line across any one area of the right temporalis muscle and the right masseter muscle, and right temporomandibular joint area.

In the embodiment, the body 110 may form a loop-shaped headgear that is worn on the top and bottom and the left and right sides of a head in the up-down direction, is worn diagonally in the right upper direction when seen from the left side of a face, has a strap structure of which both ends are detachably connected to be able to form an opening-closing type loop, and may be implemented in a left-right symmetric type.

Further, in order that the stimulating device for human 100 according to the embodiment can perform thermal therapy on the left and right temporomandibular joints, the left and right masseter muscles, and the left and right temporalis muscles, the first body 111 is worn on the left temporalis muscle, the left temporomandibular joint, and the left masseter muscle, and the second body 112 is worn on the right temporalis muscle, the right temporomandibular joint, and the right masseter muscle.

In order to wear the body 110, that is, a headgear, a first side of the first body 111 and a first side of the second body 112 may be detachably connected. For example, the first side of the first body 111 and the first side of the second body 112 may be detachably connected by various detachable couplers 113 such as Velcro, buttons, hooks, or strings, and accordingly, a portion of the circumference of the loop formed by the body 110 can be connected and disconnected.

Preferably, a detachable coupler 113 that can adjust the size of the loop formed by the body to fit to the head size of a user, in other words, a detachable coupler 113 that can adjust the gap or overlap length between the first side of the first body 111 and the first side of the second body 112, for example, a detachable coupler 113 that can tightly tighten the body 110 to fit to the head size of a user like Velcro is preferable. The body 110 forms a loop with a circumference of about 60 cm˜80 cm to fit to the face circumferences of eastern and western people.

The first body 111 and the second body 112 may be made of a raw material such as a soft material, for example, a non-conductive sheet, for example, a cloth made of natural or synthesized fiber, or a non-conductive thin synthetic resin sheet, and accordingly, the first body 111 and the second body 112 can be brought in close contact with a target area (thermal therapy object area).

A second side of the first body 111 and a second side of the second body 112 are connected to each other, and in the embodiment, the first body 111 and the second body 112 may be integrated, or the second side of the first body 111 and the second side of the second body 112 may be detachably connected by the detachable coupler described above, that is, the first body 111 and the second body 112 may be separated from each other.

In the embodiment, when the body 110 is worn on a head, the lower portions of the first body 111 and the second body 112 are integrally connected, the upper portions of the first body 111 and the second body 112 are detachably connected, and the detachable coupler 113 includes female Velcro 113a formed at any one of the upper end portion of the first body 111 and the upper end portion of the second body 112 and male Velcro 113b formed at the other one. However, the present disclosure is not limited thereto, and for example, the lower portions of the first body 111 and the second body 112 may be detachably connected and the upper portions of the first body 111 and the second body 112 may be integrally connected.

Further, as described above, the lower portions of the first body 111 and the second body 112 may be detachably connected and the upper portions of the first body 111 and the second body 112 may also be detachably connected; and at least portions of the lower portions of the first body 111 and the second body 112 and the upper portions of the first body 111 and the second body 112, preferably, both sides may be connected by the detachably coupler 113 that can adjust a loop size to fit to the head size of a user.

The electric heating thermal therapy section constituting the main thermal therapy section, etc. may be implemented using a flexible electric heating film having heating wires (e.g., cooper alloy heating wires or conductive fibers) in a synthetic resin film such as PVC, or a flexible electric heating raw material (conductive raw material) in which heating wires such as conductive fibers (conductive yarns) are applied to a raw material such as a mesh type textile or nonwoven fabric. There are various thin sheet-type electric heaters such as a heating film or a heating fabric, so these are not further described.

The sheet-type electric heater may be fixed by sewing between the lining, that is, the inside skin (part supposed to come in contact with a skin) and the outside skin (the outer surface of the body) of the body 110, or may be fixed by sewing or other methods by forming a pocket in the body 110 and putting the sheet-type electric heater into the pocket, so the method of implementing the electric heating thermal therapy section using a sheet-type electric heater may be variously changed.

Of course, the method of implementing the electric heating thermal therapy section including the main thermal therapy section 120 at the body 110 is not limited to the embodiment described above, and for example, a heating wire filament such as a conductive fiber (conductive fabric) insulatively coated directly on the body 110 may be applied.

When the body 110 is a flexible soft straps of at least two layers having an inside skin (lining) and an outside skin, the inside skin and the outside skin of the body may be made of various materials, and may be made of the same material or different materials. For example, a material made of synthetic resin such as polyurethane having flexibility may be used for the inside skin, that is, the lining of the body that is supposed to come in contact with a skin, a material that is not stained with a makeup such as so-called puffit (Hydro aircell by COREAPUFF CO. LTD) is good, and various materials such as a fiber fabric (cloth) such as common cotton or rayon, towels, sponges, or nonwoven fabrics may be use.

Further, the outside skin of the body 110 may be made of various materials such as natural or synthetic resin fabrics including cotton or rayon, and such as leather materials including artificial leather, natural leather, and vegetable leather, and as described above, it may be made of the same material as the inside skin (lining), for example, polyurethane or rayon.

The thicknesses of the lining and the outside skin are not limited. The thicknesses of the lining and the outside skin may be set the same or different within a range of about 1 mm˜20 mm, but are not limited thereto. A spongy material is good for the lining, that is, the inside skin for close contact with a skin, the sense of touch, etc., and it is preferable for the region of the body 100 that covers the temples of a head to protrude such that the surface of the inside skin is thick in order to increase close contact with a skin and transmit heat well because the temples of a head are concave.

It is preferable that the body 110 has an aligner, that is, a wearing reference of the body so that the main thermal therapy section 120 can be aligned with temporomandibular joints. That is, when the body 110 has an aligner that can be found through a sense such as the sense of sight or the sense of touch, it is possible to accurately set the wearing position of the body on the basis of the aligner. Concave cuts 114 may be formed on an edge of the regions of the body 110 covering the temporomandibular joints not to cover ears E. The cuts 114 may function as the aligner described above. In this embodiment, the cuts 114 are formed at the edges of areas covering temporomandibular joints.

The masseter muscle thermal therapy section 131 may include a left masseter muscle thermal therapy section 131a disposed at the first body 111 to perform thermal therapy on a left masseter muscle and a right masseter muscle thermal therapy section 131b disposed at the second body 112 to perform thermal therapy on a right masseter muscle.

The lower limit of the left masseter muscle thermal therapy section 131a may extend to the lower area of a left jaw line across the left lower edge of a lower jaw; and the lower limit of the right left masseter muscle thermal therapy section 131b may extend to the lower area of a right jaw line across the right lower edge of a lower jaw.

In more detail, the left masseter muscle thermal therapy section 131a may be provided in a left cheek area at which a left masseter muscle is positioned, that is, a region including an area covering a left lower edge of a lower jaw in a left masseter muscle area. Further, the right masseter muscle thermal therapy section 131b may be provided in a right cheek area at which a right masseter muscle is positioned, that is, a region including an area covering a right lower edge of a lower jaw in a right masseter muscle area.

In the embodiment, the first body 111 and the second body 112 are integrally connected. In more detail, the body 110 further includes a lower connecting portion 115 connecting the first body 111 and the second body 112. The lower connecting portion 115 may be made of a flexible material such as a flexible fabric called span, whereby it can be elongated to fit to the head size of a user. Accordingly, in the embodiment, the first sides of the first body and the second body 112 are connected by the detachable coupler 113, and the second sides of the first body and the second body 112 are connected by the lower connecting portion 115.

The lower connecting portion 115 may form a non-heat generating region between the lower limit of the left masseter muscle thermal therapy section 131a and the lower limit of the right masseter muscle thermal therapy section 131b across the bottom of the pogonion, that is, a chin strap crossing the bottom of the jaw. The lengths of the first body 111 and the second body 112 may be different from each other.

Further, the temporalis muscle thermal therapy section 132 may include a left temporal thermal therapy section 132a disposed at the first body 111 to perform thermal therapy on a left temporalis muscle and a right temporal thermal therapy section 132b disposed at the second body 112 to perform thermal therapy on a right temporalis muscle.

Further, the main thermal therapy section 120 may include a first electric heating section 121 disposed at the first body 111 to perform thermal therapy on the left temporomandibular joint and a right electric heating section 122 disposed at the second body 112 to perform thermal therapy on the right temporomandibular joint.

Accordingly, the first electric heating section 121 is formed in the region between the left masseter muscle thermal therapy section 131a and the left temporal thermal therapy section 132a, and the left temporal thermal therapy section 132a is formed in the region over the first electric heating section 121.

Further, the second heating section 122 is formed in the region between the right masseter muscle thermal therapy section 131b and the right temporal thermal therapy section 132b, and the right temporal thermal therapy section 132b is formed in the region over the second electric heating section 122. In the embodiment, the first electric heating section 121, the left masseter muscle thermal therapy section 131a, and the left temporal thermal therapy section 132a are symmetric to the second electric heating section 122, the right masseter muscle thermal therapy section 131b, and the right temporal thermal therapy section 132b.

The main thermal therapy section 120 is electrically connected to the controller 140 to control the operation of the main thermal therapy section 120, and as in the embodiment, when the head muscle thermal therapy section 130 such as the masseter muscle thermal therapy section or the temporalis muscle thermal therapy section is also an electric heating thermal therapy section, the head muscle thermal therapy section 130 may also be electrically connected to the controller 140 and controlled by one controller 140.

The embodiment has a structure in which the main thermal therapy section 120 and the head muscle thermal therapy section 130 are connected to each other, for example, in series, electricity is simultaneously supplied to the main thermal therapy section 120 and the head muscle thermal therapy section 130, and the main thermal therapy section 120 and the head muscle thermal therapy section 130 may be simultaneously operated together.

The temperature and the operation time of the electric heating thermal therapy sections 120 and 130 of the body 110 may be adjusted by the controller 140, and the technique of adjusting an electric heating temperature and time has been well known in the field of manufacturing a heater using an electric heating wire, other heating devices, or electric heating wire-built in clothes, so the technique of adjusting temperature is not further described.

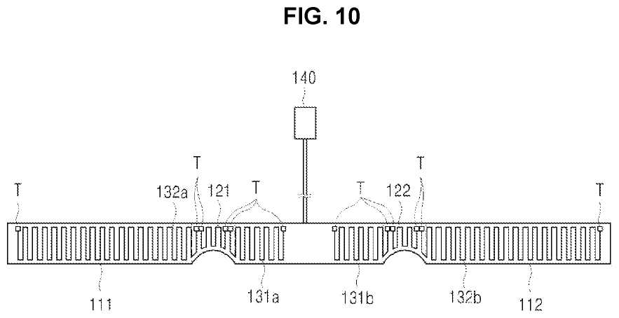

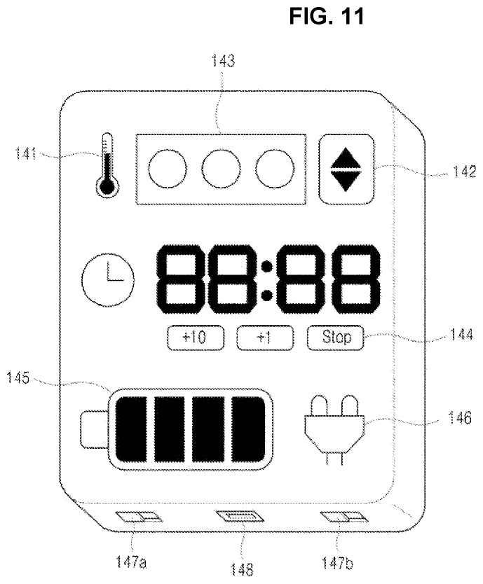

Referring to FIGS. 8 and 9, temperature can be adjusted by the controller 140, for example, in three levels (weak-medium-strong). Thermal therapy temperature for temporomandibular joints may be set at about 39° C.±5° C., thermal therapy temperature for the masseter muscles may be set at about 38° C.±5° C., and thermal therapy temperature for the temporalis muscles may be set at about 41° C.±5° C. When the upper limit is exceeded, tissues may be injured in the areas, and when the lower limit is not reached, it may be not enough to obtain the thermal therapy effect. In general thermal therapy for the temporomandibular joints, masseter muscles, and temporalis muscles, appropriate heat generation temperature may be selected in the section at which the temperature ranges of the three areas overlap. For example, the heat generation temperatures of the thermal therapy sections may be set to be adjusted step by step in the section of 39° C.±4° C.

As exemplified in FIG. 8, the controller 140 may be equipped with a temperature indicator 141, temperature control buttons 142, and a temperature intensity lamp 143. The embodiment is implemented in the type in which temperature can be adjusted in three levels, as in FIG. 9(a), but is not limited thereto, and for example, temperature may be adjusted in two level or five levels.

Further, in the embodiment, a thermal therapy time may be adjusted by a timer of the controller, as in FIG. 9(b).

Referring to FIG. 8, the controller 140 may be equipped with a stop button 144, and accordingly, stopping in operation is possible. Further, the controller 140 may be equipped with a battery level indicator 146 and a lamp 146 showing whether a charging line is connected (charging state), and may be equipped with a charging line connection port 147 and a power switch 148, that is, an On/Off switch. A battery may be disposed in the controller 140. Of course, the controller 140 may be implemented in a wireless type, that is, a remote controller type, and in this case, the stimulating device for human 100 may be connected directly to an external power source and supplied with power, or a chargeable or a replaceable (disposable) battery and a control signal receiver may be mounted on the body 110 or a neck stimulation band to be described below.

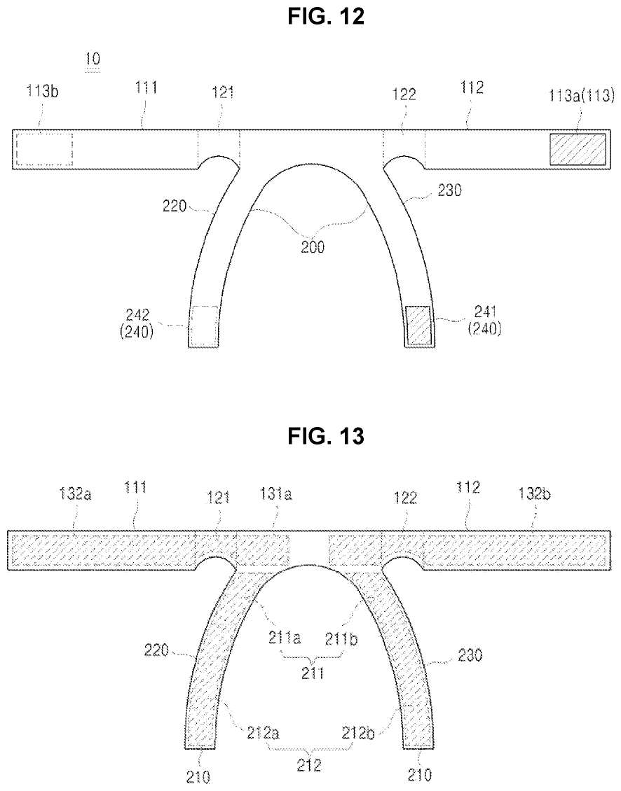

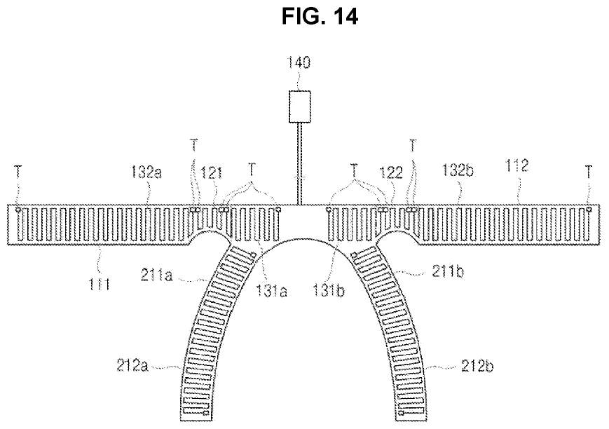

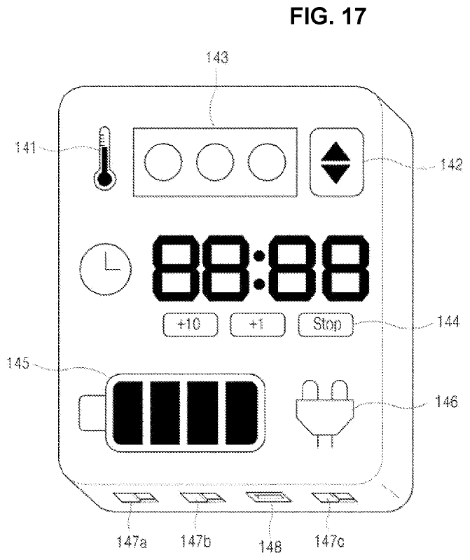

Referring to FIGS. 10 and 11, the main thermal therapy section 120 and the head muscle thermal therapy section 130 may be individually controlled, and the masseter muscle thermal therapy section 131 and the temporalis muscle thermal therapy section 132 may be connected in series or in parallel and simultaneously controlled. The controller 140 for individually (independently) controlling the main thermal therapy section 120 and the head muscle thermal therapy section 130 may be equipped with a muscle thermal therapy power switch 147a and a joint (temporomandibular joint) thermal therapy power switch 147b. For reference, the reference character “T” shown in FIGS. 3, 10, and 14 is a connection point at which wires are connected.

Of course, the masseter muscle thermal therapy section 131 and the temporalis muscle thermal therapy section 132 may be individually controlled. A method of configuring a control circuit that individually controls the operation of several separate electric heating regions has been well known in the field of control, so it is not further described.

The stimulating device for human according to the present disclosure may be implanted in the type of a device further including a neck stimulation band 200 for performing thermal therapy on neck muscles to be described above together with the components, for example, a stimulating device for human for stimulating both temporomandibular joints and a neck, in more detail, a head-wearable electric thermal therapy device.

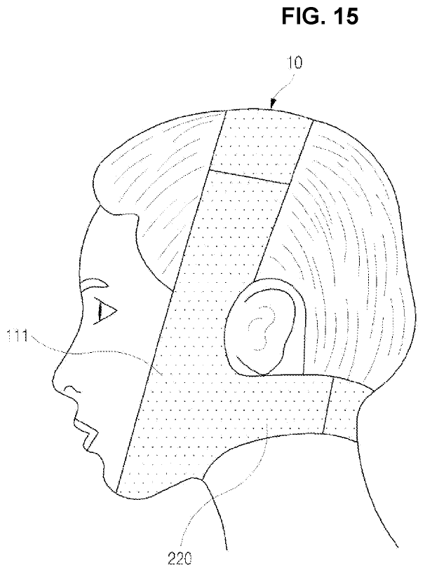

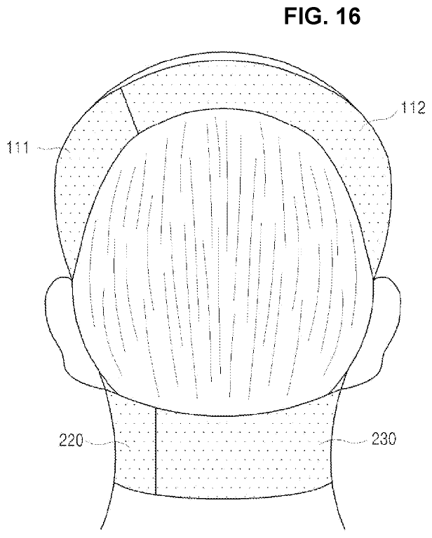

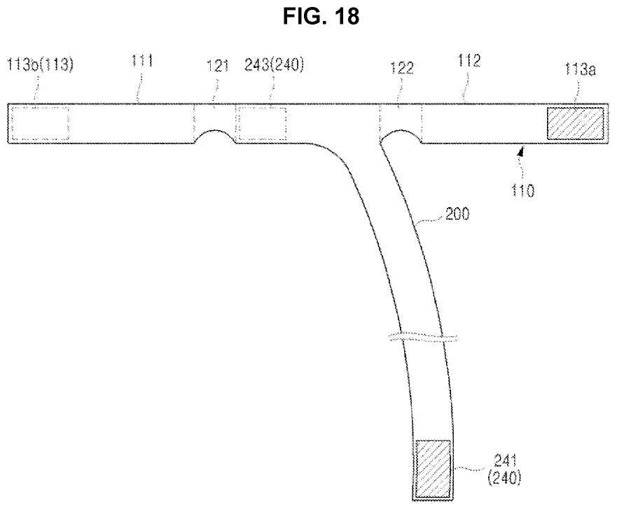

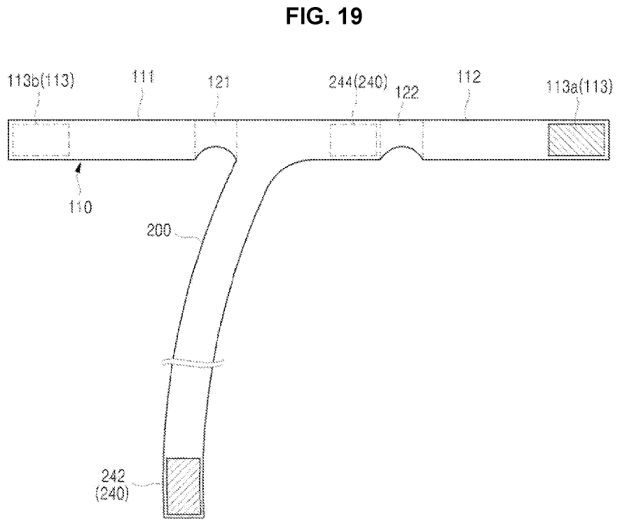

Referring to FIGS. 12 to 16, a second embodiment 10 of the stimulating device for human according to the present disclosure further includes the neck stimulation band 200 for performing thermal therapy on neck muscles in comparison to the first embodiment 100.

In other words, the stimulating device for human according to the second embodiment of the present disclosure, which is a device having a function of thermal therapy for the temporomandibular joints and a function of thermal therapy for neck muscles, includes a body 110 supposed to be worn on a head, an electric heating thermal therapy section 120 disposed at the body 110 to be able to perform thermal therapy on the temporomandibular joints and generating heat using electricity, and a neck stimulation band 200 for performing thermal therapy on neck muscles.

The body 110 may further includes a heat muscle thermal therapy section 130, for example, the masseter muscle thermal therapy section 131 and/or the temporalis muscle thermal therapy section 132 besides the electric heating thermal therapy section 120 (main thermal therapy section) described in the first embodiment, and the description in the first embodiment may be equally applied for the thermal therapy section including the body 110 and the main thermal therapy section 120, so they are not limited.

The neck stimulation band 200 has a neck thermal therapy section 210 for performing thermal therapy on muscles crossing the neck, and is disposed under the body 110 to be able to be worn on a neck.

The neck stimulation band 200 may be connected to the lower portion of the body 110, as in the embodiment. Of course, it may be possible to configure a stimulating device for human in which the body 110 and the neck stimulation band 200 are separated and worn on a head and a neck, respectively, and the body 110 and the neck stimulation band 200 may be integrated or may be detachably connected to each other.

The neck stimulation band 200 may include a left stimulation band 220 that can be worn on the left side of a neck and a right stimulation band 230 that can be worn on the right side of a neck. In order to put the neck stimulation band on a neck, a first side of the left stimulation band 220 and a first side of the right stimulation band 230 may be detachably connected, thereby forming an opening/closing type loop that is wound around a neck.

As a detachable coupler 240 detachably connecting the first side of the left stimulation band 220 and the first side of the right stimulation band 230, various detachable couplers that are wound around a neck and can adjust the size of a loop formed by the neck stimulation band to fit to the circumference of a neck such as Velcro, buttons, hooks, or strings described above in the first embodiment may be applied.

For example, the detachable coupler of the neck stimulation band may include a female Velcro 241 disposed at any one of the first side of the left stimulation band 220 and the first side of the right stimulation band 230, and male Velcro 242 disposed at the other side. The neck stimulation band 200 forms a loop with a circumference of about 36 cm˜50 cm to fit to the neck circumferences of eastern and western people.

The left stimulation band 220 may be connected to a lower side (left side) of the body 110 and may extend, and the right stimulation band 230 may be connected to another lower side (right side) of the body 110. The electric heating thermal therapy sections 120 and 130 disposed at the neck thermal therapy section 210 and the body may be simultaneously or individually operated.

The neck thermal therapy section 210 can generate heat for thermal therapy that is applied to at least one of a sternocleidomastoideole (SCM muscles) and splenius capitis muscle of a neck. The neck thermal therapy section 210 in the embodiment may be an electric heating thermal therapy section, that is, a heater generating heat using electricity, and may include a first neck thermal therapy section 211 for performing thermal therapy on the sternocleidomastoideole and a second neck thermal therapy section 212 for performing thermal therapy on the splenius capitis muscle. The first neck thermal therapy section 211 and the second neck thermal therapy section 212 may be individually or simultaneously controlled.

As a more detailed example, the first neck thermal therapy section 211 may include a first left thermal therapy section 211a disposed at the left stimulation band 220 at the left side of a neck from a center line to perform thermal therapy on sternocleidomastoideole, and a first right thermal therapy section 211b disposed at the right stimulation band 220 at the right side of a neck from the center line to perform thermal therapy on sternocleidomastoideole.

Further, the second neck thermal therapy section 212 may include a second left thermal therapy section 212a disposed at the left stimulation band 220 at the left side of a neck from the center line to perform thermal therapy on splenius capitis muscle, and a second right thermal therapy section 212b disposed at the right stimulation band 220 at the right side of a neck from the center line to perform thermal therapy on splenius capitis muscle.

The sternocleidomastoideole and the splenius capitis muscle contribute to stabilizing the position of a jaw, and when a pain is generated at this area, a pain (Heterotopic Pain) may be generated at other positions such as the masseter muscles and the temporalis muscles rather than the area of origin. When thermal therapy is applied to only the masseter muscle and/or the temporalis muscle area at which a pain is felt, the symptom may not get better, and it is possible to attenuate a pain by performing thermal therapy on the sternocleidomastoideole and the splenius capitis muscle through the neck stimulation band 200.

The thermal therapy temperatures for the sternocleidomastoideole and the splenius capitis muscle may be set the same as the thermal therapy temperature for the masseter muscle or the masticatory muscles, and the left stimulation band 220 and the right stimulation band 230 may be different in length.

In the second embodiment of the present disclosure, the electric heating thermal therapy sections 120 and 130 of the body and the neck thermal therapy section 210 are simultaneously operated, and when the controller is powered and the intensity of temperature and operation time are set, a current flows simultaneously to the electric heating thermal therapy sections 120 and 130 of the body and the neck thermal therapy section 210, whereby the electric heating thermal therapy sections 120 and 130 of the body and the neck thermal therapy section 210 simultaneously generate heat. It is preferable to set thermal therapy time for temporomandibular joints, head muscles, and neck muscles as 30 minutes or less per one time.

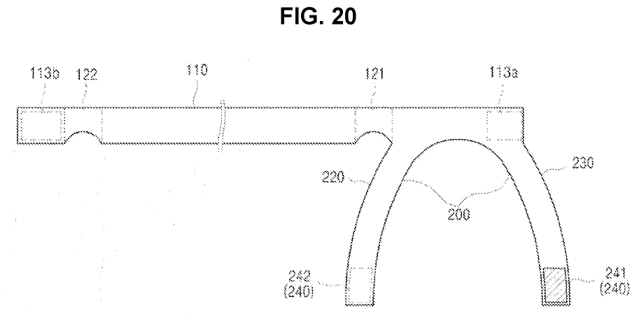

Referring to FIG. 17, in the second embodiment of the present disclosure, the main thermal therapy section 120, the head muscle thermal therapy section 130, and the neck thermal therapy section 210 for performing thermal therapy on temporomandibular joints may be individually controlled, and muscle thermal therapy sections such as the head muscle thermal therapy section 130, the neck thermal therapy section 210, etc. for performing thermal therapy on muscles may be simultaneously operated. The controller 140 for individually (independently) controlling the main thermal therapy section 120 and the muscle thermal therapy sections 130 and 210 may be equipped with a head muscle thermal therapy power switch 147a for performing thermal therapy on the masseter muscles and the temporalis muscles, a joint (temporomandibular joint) thermal therapy power switch 147b, and a neck muscle thermal therapy power switch 147c.

Of course, the masseter muscle thermal therapy section 131, the temporalis muscle thermal therapy section 132, the sternocleidomastoideole thermal therapy section (first neck thermal therapy section), and the splenius capitis muscle thermal therapy section (second neck thermal therapy section) may be individually controlled. A method of configuring a control circuit that individually controls the operation of several separate electric heating regions, as described above, has been well known in the field of control, so it is not further described.

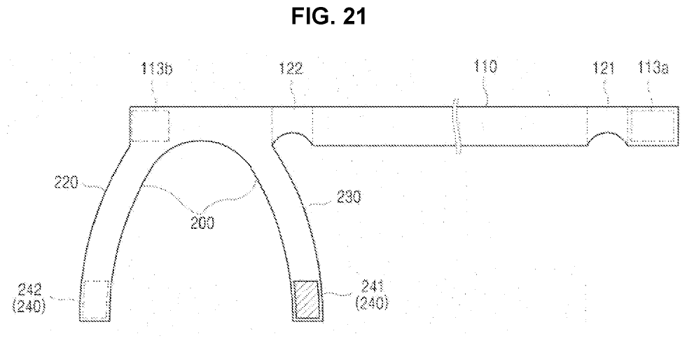

FIGS. 18 to 21 are views schematically showing other embodiments of the stimulating device for human for temporomandibular joints and the neck in a plane.

FIGS. 18 and 19 exemplify a shape in which one branch of neck stimulation band 200 extends from the stimulation body 110 having the first body 111 and the second body 112 at both sides from a center line. The second end of the neck stimulation band 200 may be detachably connected to the body 110 by various detachable couplers 240 (241, 242, 243, and 244) such as Velcro, buttons, and hooks. For example, female Velcro may be formed at the body 110 and male Velcro may be formed at the second end portion of the neck stimulation band 200.

Further, FIGS. 20 and 21 show a shape in which one end portion of the body 110 equipped with an electric heating thermal therapy section including the main thermal therapy sections 121 and 122 for performing thermal therapy on temporomandibular joints is connected to neck stimulation bands 200 (220 and 230) separated left and right, and the body elongates in one direction, that is, as one branch. The second end of the body 110 may be detachably connected to the body 110 at the joint between the neck stimulation band 200 and the body by various detachable couplers 113a and 113b such as Velcro, buttons, or hooks.

The embodiments of the stimulating device for human, as described above, may be implemented in the type of an electric thermal therapy device, that is, a head-wearable electric thermal therapy device. Accordingly, the body 110 described in various embodiments of the stimulating device is configured to be worn on a head in contact with the head, so it may be referred to as a headgear, and the embodiments may be referred to as headgear-type electric thermal therapy devices.

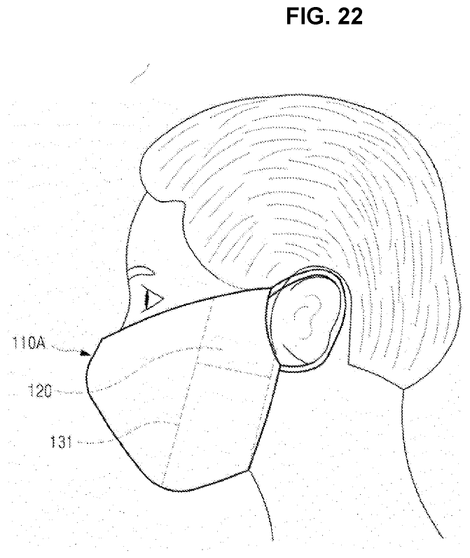

FIG. 22 is a view showing another embodiment of a stimulating device for human including a mask-shaped body, that is, a mask type headgear 110A, and an electric heating thermal therapy section disposed at the headgear. In the embodiment, the electric heating thermal therapy section includes an electric heating temporomandibular joint thermal therapy section 120 and may further include an electric heating masseter muscle thermal therapy section 131.

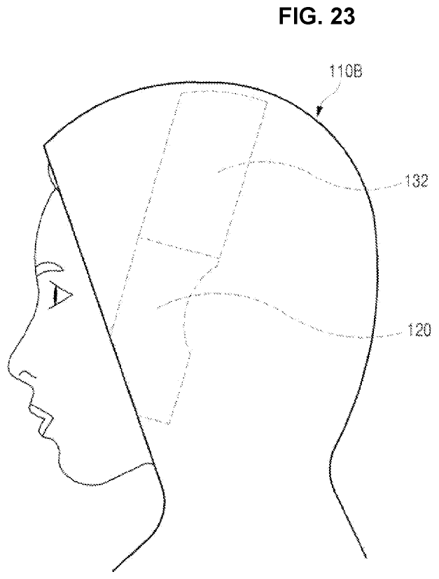

Further, FIG. 23 is a view showing another embodiment of a stimulating device for human including a hood type body, that is, a hood type headgear 110B, and an electric heating thermal therapy section disposed at the headgear. The hood type headgear 110B is configured to be worn on a head, covers temporomandibular joint areas and temporalis muscle areas, and may have a temporomandibular joint thermal therapy section 120 and a temporalis muscle thermal therapy section 132. Further, the portions corresponding to ears of the sides of the headgear may be open. Of course, the hood type headgear 110B may be formed in a size that can cover masseter muscle areas, and the rear of the headgear may be open such that the occipital area is exposed.

The embodiments of various stimulating device for humans described above may further have other functions of stimulation for human besides the function for thermal therapy. For example, a light source for various optical therapies such as wrinkle care or skin stimulation may be disposed at the body or the neck stimulation band of the stimulating devices described above, a vibrator for applying massage stimulation, for example, vibration stimulation to a head or a neck may be disposed, and particularly, since the embodiments are operated by electricity, various electric stimulating devices based on electric energy may be added to the body.

The embodiments of the present disclosure described above may be manufactured in an individual-fit type to fit to the face sizes and skeletons of users. Of course, the embodiments may be manufactured in various sizes, for example, three sizes of small, medium, and large so that users can select a stimulating device for human suitable for their face/neck sizes and skeletal shapes. Further, the shape of a strap constituting the body and the neck stimulation band proposed in the drawings was schematically exemplified. Accordingly, the headgear type body and neck stimulation band may be designed in appropriate shapes supposed to be worn on predetermined areas (the temporomandibular joints, the masseter muscles, and the temporalis muscles) of a head and predetermined areas (the sternocleidomastoideole and the splenius capitis muscle) of a neck.

Although preferred embodiments of the present disclose were described above, it would be apparent to those skilled in the art that the present disclosure may be achieved in other specific types without departing from the scope or spirit other than the embodiments described above.

Accordingly, the embodiments described above should be considered as being exemplifying rather than limiting, so the present disclosure may be changed within the range of the claims and the equivalent range without being limited to the above description.

### INDUSTRIAL APPLICABILITY

An objective of the present disclosure can be used in various industrial fields such as a medical equipment field, a medical prosthesis field, or a health assistant field that manufactures a head-wearable body stimulating device, for example, a headgear-type electric thermal therapy device that has a function for thermal therapy, particularly, a function for thermal therapy to temporomandibular joints, can be worn on the head, and can perform thermal therapy on temporomandibular joints using electrical heat.

1. A stimulating device for human having a function for thermal therapy to temporomandibular joints, the stimulating device comprising:
a body configured to be worn on a head;
an electric heating main thermal therapy section disposed at the body to be able to perform thermal therapy on temporomandibular joints and generating heat using electricity; and
at least one head muscle thermal therapy section of a masseter muscle thermal therapy section and a temporalis muscle thermal therapy section disposed at the body together with the main thermal therapy section.

2. (canceled)

3. The stimulating device of claim 1, wherein the head muscle thermal therapy section generates heat using electricity.

4. The stimulating device of claim 3, wherein the main thermal therapy section and the head muscle thermal therapy section can be individually controlled.

5. The stimulating device of claim 1, wherein the head muscle thermal therapy section includes both of the masseter muscle thermal therapy section and the temporalis muscle thermal therapy section, and
the masseter muscle thermal therapy section and the temporalis muscle thermal therapy section can be individually controlled.

6. The stimulating device of claim 1, wherein the body includes:
a first body configured to be worn on a left side of a head from a center line; and
a second body configured to be worn on a right side of a head from the center line.

7. The stimulating device of claim 6, wherein the masseter muscle thermal therapy section includes:
a left masseter muscle thermal therapy section disposed at the first body to perform thermal therapy on a left masseter muscle; and
a right masseter muscle thermal therapy section disposed at the second body to perform thermal therapy on a right masseter muscle,
wherein the temporalis muscle thermal therapy section includes:
a left temporal thermal therapy section disposed at the first body to perform thermal therapy on a left temporalis muscle; and
a right temporal thermal therapy section disposed at the second body to perform thermal therapy on a right temporalis muscle.

8. The stimulating device of claim 7, wherein a lower limit of the left masseter muscle thermal therapy section extends to a lower area of a left jaw line across a left lower edge of a lower jaw; and a lower limit of the right left masseter muscle thermal therapy section extends to a lower area of a right jaw line across a right lower edge of a lower jaw.

9. The stimulating device of claim 8, wherein the body further includes a connecting portion going through a pogonion while connecting the first body and the second body, and forming a non-heat generating region between the lower limit of the left masseter muscle thermal therapy section and the lower limit of the right masseter muscle thermal therapy section.

10. (canceled)

11. The stimulating device of claim 6, wherein the main thermal therapy section includes:
a first electric heating section disposed at the first body to perform thermal therapy on a left temporomandibular joint; and
a second electric heating section disposed at the second body to perform thermal therapy on a right temporomandibular joint.

12. The stimulating device of claim 7, wherein the first body can be worn on the left side of the head from the center line across any one area of the left temporalis muscle and the left masseter muscle, and a left temporomandibular joint; and
the second body can be worn on the right side of the head from the center line across any one area of the right temporalis muscle and the right masseter muscle, and a right temporomandibular joint.

13. The stimulating device of claim 6, wherein a first side of the first body and a first side of the second body are detachably coupled so that the body is worn on a head.

14. The stimulating device of claim 6, wherein the first body and the second body are made of a flexible material.

15. The stimulating device of claim 1, further comprising a neck stimulation band having a neck thermal therapy section for performing thermal therapy on muscles crossing a neck, and disposed under the body to be able to be worn on a neck.

16. The stimulating device of claim 15, wherein the neck stimulation band is connected to the lower portion of the body.

17. The stimulating device of claim 15, wherein the neck stimulation band includes:
a left stimulation band being able to be worn on a left side of the neck; and
a right stimulation band being able to be worn on a right side of the neck.

18. The stimulating device of claim 17, wherein a first side of the left stimulation band and a first side of the right stimulation band are detachably connected so that the neck stimulation band is worn on a neck.

19. The stimulating device of claim 15, wherein the neck thermal therapy section can generate heat to perform thermal therapy on at least one of a sternocleidomastoideole and a splenius capitis muscle of the neck.

20. The stimulating device of claim 19, wherein the neck thermal therapy section includes a first neck thermal therapy section for performing thermal therapy on the sternocleidomastoideole, and a second neck thermal therapy section for performing thermal therapy on the splenius capitis muscle.

21. (canceled)

22. An electric thermal therapy device having a function for thermal therapy to temporomandibular joints, the electric thermal therapy device comprising:
a headgear configured to be worn on a head;
a first electric heating section disposed at a first side of the headgear to be able to perform thermal therapy on a left temporomandibular joint at a left side of the head from a center line of the head, and generating heat using electricity;
a second electric heating section disposed at a second side of the headgear to be able to perform thermal therapy on a right temporomandibular joint at a right side of the head, and generating heat using electricity;
at least one electric heating left head muscle thermal therapy section of a left masseter muscle thermal therapy section and a left temporal thermal therapy section provided with the first electric heating section at the first side of the headgear; and
at least one electric heating right head muscle thermal therapy section of a right masseter muscle thermal therapy section and a right temporal thermal therapy section provided with the second electric heating section at a second side of the headgear.

23. (canceled)

24. The electric thermal therapy device of claim 22, further comprising a neck stimulation band having a neck thermal therapy section for performing thermal therapy on muscles at a neck, and disposed under the headgear to be able to be worn on the neck.
